(19) United States
(12) Patent Application Publication
(10) Pub. No.: US 2018/0280620 A1
(43) Pub. Date: Oct. 4, 2018

(54) SHIVERING ADJUSTMENT INFUSION SYSTEM AND METHOD

(71) Applicant: SEIRATHERM GMBH, Herzogenaurach (DE)

(72) Inventors: Thomas REICHTHALHAMMER, Polling (DE); Matthias ROTH, Giggenhausen (DE)

(73) Assignee: SEIRATHERM GMBH, Herzogenaurach (DE)

(21) Appl. No.: 15/764,688

(22) PCT Filed: Sep. 29, 2016
(86) PCT No.: PCT/EP2016/073246

§ 371 Date: Mar. 29, 2018

(30) Foreign Application Priority Data
Sep. 30, 2015 (EP) 15187807.1

(51) Int. Cl. A61M 5/172; A61M 5/44; A61M 5/168

(52) U.S. Cl. CPC A61M 5/1723; A61M 5/445; A61M 5/1407; A61M 2005/1726; A61M 5/16877

(57) ABSTRACT

The present invention is directed to an adjustment device for controlling infusion fluid in particular for temperature regulation therapy such as hypothermia treatment. The invention also concerns a hypothermia device, system or method that is adapted for shivering control. The device comprises a control unit is configured to receive input signals indicating level of shivering and to provide output signals indicating one or more recommendation for therapy based on said received input signals.

### FIELD

The invention is generally directed to devices and methods for controlling infusion fluid in particular for temperature regulation therapy such as hypothermia treatment. The invention also concerns a hypothermia and optional hyperthermia device, system or method that is adapted to minimize or suppress shivering.

### BACKGROUND

Hypothermia is usually known as a condition in which the body's core temperature drops below that required for normal metabolism and body functions. This is generally considered to be less than about 35.0° C. (about 95.0° F.). Characteristic symptoms depend on the temperature. Targeted temperature management (TTM) previously known as therapeutic hypothermia or protective hypothermia is an active treatment that aims to achieve and maintain a specific body temperature in a person for a specific duration of time in an effort to improve health outcomes. This is done in an attempt to reduce the risk of tissue injury from lack of blood flow. Periods of poor blood flow may be due to cardiac arrest or the blockage of an artery by a clot such as may occur during a stroke. Targeted temperature management improves survival and brain function following resuscitation from cardiac arrest. Evidence supports its use following certain types of cardiac arrest in which an individual does not regain consciousness. Targeted temperature management can advantageously prevent brain injury by several methods including decreasing the brain's oxygen demand, reducing the proportion of neurotransmitters like glutamate, as well as reducing free radicals that might damage the brain. The lowering of body temperature may be accomplished by many means including the use of cooling blankets, cooling helmets, cooling catheters, ice packs and ice water lavage.

Medical events that targeted temperature management may effectively treat fall into five primary categories: neonatal encephalopathy, cardiac arrest, ischemic stroke, traumatic brain or spinal cord injury without fever, and neurogenic fever following brain trauma.

Applicants' prior application WO2012143479, incorporated herein, provides a useful general description of an apparatus for temperature therapy.

Document U.S. Pat. No. 7,896,834 B2 discloses a pump system selectably controlling the temperature, flow rate, flow volume, and flow pressure of a fluid being infused into a patient's body. The apparatus comprises means for delivering a predetermined volume or halting device operation when an excessive volume has been infused.

Document U.S. Pat. No. 8,672,884 B2 discloses methods for introducing fluids into a body cavity for hypothermic treatment. In one embodiment of the invention, at least one of the rate or volume of infusate is configured to increase a mean patient blood pressure. In another embodiment, the infusion parameter is at least one of a flow rate, a pressure, a total infused volume, an inflow duty cycle or a hypothermic solution temperature.

An important adverse effect of the therapeutic temperature modulation is shivering. Not only is shivering uncomfortable for the patient, it also results in sharp increases in resting energy expenditure (REE) and in the systemic rate of oxygen consumptions, which leads to the unwanted increase in body temperature.

US Patent Application No. 20050064186 relates to a treatment of patient for reducing shivering and involves disposing cooling heat exchange pad(s) against skin of patient, exchanging heat with patient using pad to lower body temperature of a patient, and administering an anti-shivering medication.

European Patent Application No. 2008849920 concerns a system and method that employ a monitoring device to monitor at least one patient physiological response to a change in temperature of the patient, e.g. pursuant to induced hypothermia therapy, wherein a monitoring signal is provided by the monitoring device. In turn, an output, e.g. a visual and/or auditory output, may be provided to a user indicative of at least one measure of a patient's response to the change in temperature.

### SUMMARY OF THE INVENTION

Therapeutic hypothermia may be useful in various circumstances including stroke. However, core body temperature is normally tightly regulated. Even mild hypothermia in conscious subjects thus provokes vigorous thermoregulatory defenses which are potentially harmful in fragile patients. The major difficulty with induction of therapeutic hypothermia is that it may provoke vigorous thermoregulatory defenses, particularly vasoconstriction and shivering. Shivering is a bodily function in response to early hypothermia or feeling cold in warm-blooded animals. When the core body temperature drops, the shivering reflex is triggered to maintain homeostasis. Skeletal muscles begin to shake in small movements, creating warmth by expending energy. Shivering can also be a response to a fever, as a person may feel cold. The primary motor center for shivering is located in the posterior hypothalamus near the wall of the third ventricle. This area is normally inhibited by signals from the heat center in the anterior hypothalamic-preoptic area but is excited by cold signals from the skin and spinal cord. The center becomes activated when the body temperature falls even a fraction of a degree below a critical temperature level. Increased muscular activity results in the generation of heat as the main intended utilized for warmth. Not only do vasoconstriction and shivering slow onset of hypothermia, but they are associated with hypertension, tachycardia, and sympathetic nervous system activation. Shivering is involuntary, and augments metabolic heat production. The shivering threshold is normally about 35.5° C., about 1° C. below the vasoconstriction threshold. Shivering can double metabolic heat production over long periods. To the extent that shivering increases metabolic rate, it diminishes the efficacy of applied cooling, and thus slows induction of therapeutic hypothermia. Furthermore, shivering is potentially harmful, because it is associated with hypertension and sympathetic nervous system activation. Thus, there is a need to contain or minimize the level of shivering of the patient during treatment.

The problem underlying the present invention is to provide an improved device and methods for controlling infusion fluid. The problem is solved by the subject matter of the present invention exemplified by the description and the claims.

Further features and advantages of the present disclosure will become apparent from the following detailed description.

The present invention provides an improved device for controlling and managing administration of infusion fluid that takes account of the adverse effect of shivering caused by the infusion fluid. The device of the invention is adapted to receive inputs of signals which indicates the level of shivering of the patient and provides recommendations for minimizing or suppressing shivering. The input signals may be provided by a user, from an external computer system, or internally from a component of the device.

The invention provides a device for controlling and managing administration of infusion fluid for temperature regulation therapy, comprising at least one flow control unit for regulating flow rate and/or temperature of infusion fluid, and at least one control unit for receiving input signals and providing output signals, wherein the control unit is configured to receive input signals indicating the level of shivering of the patient and to provide output signals indicating one or more recommendations for therapy based on said received input signals.

The term “temperature regulation therapy” in the present application refers to a process of controlling a patient's body temperature below the normal body temperature. This can be achieved by using invasive temperature management treatments which, among others, include the infusion of cold intravenous fluids (also referred to herein as infusion fluids). Invasive temperature management treatments often require constant personnel involvement and attention to perform successfully. Moreover, certain invasive temperature management modalities have been associated with overcooling, overheating, or, more often, inadequate warming.

The infusion fluid can be any known fluids such as blood/blood derivatives, pharmacological fluids, nutritional fluids, and fluid infusion systems and/or an infusion system for infusing, e.g., saline or other balanced fluids like ringer's solution. Also the kind, shape, material and volume can vary. Any infusion fluid can be any fluid administered intravenously to a patient, such as saline solution or other type of conventional IV solution or any solution such as a blood solution, dissolved drug or the like, administered to a patient via intravenous infusion. For instance, the infusion fluid could be blood, particularly extra corporal fluids like blood, dialysis liquids or substitute liquids, more preferably an infusion liquid such as electrolyte solutions such as NaCl, Ringer solutions, or Jonosteril®. According to the present invention, the infusion of a fluid may be used for controlling the body temperature.

The device according to the present invention may comprise a flow control unit. A flow control unit in general refers to a device or arrangement that enables the device to actively maintain a certain flow rate, i.e. a pumping mechanism, which allows controlled, variable flow rate of the infusion fluid. A flow control unit may regulate the flow rate and temperature of infusion fluid. The pumping mechanism can comprise a pump of any kind available in the market, such as a peristaltic pump, piston pumps etc. The pump can be adapted to deliver the infusion fluid continuously and/or intermittently and/or sequentially, the latter preferably on the basis of pulses and intermediate pauses with volumes during the pulses of between 1 ml to 50 ml.

Homeostatic mechanisms maintain body temperature between 36-37° C. They include vasodilation, vasoconstriction and shivering. When the skin receives a continuous cold sensation, motor neurons are stimulated, creating of a shivering response in the muscles of the body. Shivering generates heat by creating friction among the muscle spindles and generating heat during involuntary contractions. This movement is a rhythmic tremor of skeletal muscle groups that consists of oscillatory movements. Shivering progresses from the masseter muscle, to neck and thorax, and finally the trunk and extremities. In general, this response is activated when the body temperature decreases 1° C. below the threshold for vasoconstriction, usually between a core body temperature of 34° C.-36° C. The threshold is different for each person, and the shivering response depends on body mass and gender, as well as other factors like medications, time of the day and menstrual cycles. Physiologically, shivering leads to an increase in oxygen consumption, CO2 production, and an increase in circulating catecholamine concentrations, causes systemic vasoconstriction that further reduces peripheral perfusion and, consequently, increases thermal isolation of core and peripheral tissues. Shivering is also associated with a significant decrease of brain tissue oxygenation (PbtO2).

Level of shivering could be assessed in many ways, including observation of piloerection (erection of hair on the skin of the arms and legs), tactile confirmation of a vibration in the mandible and neck region, visualization of tremors, and measurements with electrical signals of muscle activity, such as electromyography (EMG). It is preferred that the detection of shivering is based on clinically accepted standard, such as the bedside shivering assessment scale BSAS (Badjatia et al., Metabolic impact of shivering during therapeutic temperature modulation: the Bedside Shivering Assessment Scale. Stroke 2008; 39:3242-3247). The BSAS score may be measured by palpating the temples and masseters, neck and shoulders, pectoralis muscles, biceps, and quadriceps. Current BSAS is a 4-point scale, which rates shivering as absent, or mild, moderate, or severe (Table 1). The 4-point scale was validated against resting energy expenditure, oxygen consumption, and carbon dioxide production as measured by indirect calorimetry. The BSAS score provided an accurate representation of the initial and ongoing metabolic stress that occurred during shivering. A BSAS score of 2 to 3 was associated with a resting energy expenditure of 2303 to 3686 kcal/d compared with patients with a BSAS score of 0 to 1, who expended approximately 1390 to 1730 kcal/d.16 These results indicate that the BSAS can be used as a reliable tool for determining the metabolic consequences of shivering.

TABLE 1

| Score | Definition |
|---|---|
| 0 | None: no shivering noted on palpation of the masseter, neck, |
| | or chest wall |
| 1 | Mild: shivering localized to the neck and/or thorax only |
| 2 | Moderate: shivering involves gross movement of the upper |
| | extremities (in addition to neck and thorax) |
| 3 | Severe: shivering involves gross movements of the trunk and |
| | upper and lower extremities |

The device according to the present invention comprises a control unit configured to receive input signals and to provide output signals.

The control unit may be configured to receive input signals indicating the level of shivering of the patient. The control unit may additionally be configured to receive input data indicating body temperature, including core body temperature or body surface temperature of patient and/or the desired therapeutic body temperature of the patient.

The control unit generally comprises a processor and a memory, for receiving and storing signal data, and for storing and executing programs for processing the received signals and controlling the flow control unit, and providing any suitable output signals and/or information that may be desired to implement. The control unit provides output signals that indicate at least recommendations for the therapy. Based on the input information received, the device provides recommendations to a user that include information as to which action can be taken in response to excessive shivering.

In one preferred embodiment, the control unit is configured to receive input signals from at least one external computer system. This is particularly useful when used in hospitals using electronic patient journal systems that store and make available patient data such as biosignals (blood pressure, pulse, hemoglobin values, etc.), data from analyzed patient samples, and data concerning administered therapy, including but not limited to medicaments and fluids that have been or are being administered. It will be appreciated that the control unit is in some embodiments able to receive input directly from such at least one external computer system, with a suitable program interface to query the external system for the desired data. In other embodiments, the control unit prompts a user to feed the unit with desired data from such an external computer system, manually, or by entering data files in suitable format.

In addition, a control unit according to the present invention may be configured to provide output signals, also termed herein as compensation signals, which provide one or more suggestions or recommendations intended for decreasing the level of shivering. For example, the control unit can be configured to provide a signal to the flow control unit, such as to reduce flow rate or increase temperature of the infusion fluid.

Alternatively, the control unit may provide an output signal to the flow control unit to maintain the flow rate and/or the temperature of the infusion fluid, and additionally provide one or more recommendations to administer anti-shivering medication and/or an instruction to raise surface temperature of the patient.

Some common infusion fluids are defined below in a non-limiting list.

TABLE 2

| | Na⁺ | K⁺ | Ca⁺ | Mg⁺ | Cl⁻ | Lactate⁻ | Acetate⁻ | Osmolarity |
|---|---|---|---|---|---|---|---|---|
| | mmol/L | mmol/L | mmol/L | mmol/L | mmol/L | mmol/L | mmol/L | (mOsm/L) |
| NaCl | 154 | — | — | — | 154 | — | — | 308 |
| 0.9% | | | | | | | | |
| Ringer's | 147 | 4.0 | 2.3 | — | 156 | — | — | 309 |
| soln | | | | | | | | |
| Ringer- | 125-134 | 4.0-5.4 | 0.9-2.0 | — | 106-117 | 25-31 | — | 262-293 |
| lactate | | | | | | | | |
| soln | | | | | | | | |
| Ringer- | 130 | 5.4 | 0.9 | 1.0 | 112 | — | 27 | 276 |
| acetate | | | | | | | | |
| soln. | | | | | | | | |

There are slight variations for the exact composition for some of the above mentioned solutions (such as Ringer's solution, Ringer's lactate solution, etc.) as supplied by different manufacturers, thus such terms should not be equated with one precise formulation.

It is envisioned that the recommendation or suggestion comprises an instruction to administer one or more anti-shivering medication. As used herein, the term anti-shivering medication is intended to mean any biologically active agent or drug or combination of agents or drugs that is administered to a patient for the purpose of reducing shivering.

Preferred anti-shivering medications include dopamine receptor blockers (neuroleptic drugs and dopamine receptor agonists), opioids including morphine, opioid receptor agonists and antagonists such as kappa (κ) opioid receptor agonists, mu (μ) opioid receptor antagonists, opioid agonist-antagonist analgesic drugs, and serotonin receptor agonists such as serotonin HT1a receptor agonists, and pharmaceutically acceptable salts of any of the aforementioned agents. Combinations of these anti-shivering medications are also contemplated.

Anti-shivering agents or medications are known in the art. There are numerous dopamine receptor blockers (neuroleptic drugs and dopamine receptor antagonists), or mixtures thereof that are suited for use in the methods of the invention. Suitable dopamine receptor blockers include phenothiazines, in particular those having one or more aliphatic, piperidine and piperazine groups such as are described by Adler, et al., U.S. Pat. No. 4,758,562, the disclosure of which is incorporated herein by reference. Exemplary phenothiazines include, by way of illustration and not limitation, aliphatic, halogenated phenothiazines such as chlorpromazine, triflupromazine, and the like; piperidine phenothiazines such as thioridazine, mesoridazine, piperacetazine, and the like; piperazine phenothiazines such as fluphenazine, trifluoperazine, acetophenazine, carphenazine, fluphenazine, perphenazine, prochlorperazine and the like.

Anti-shivering medications which can be used in the art include for example but not limited to opiates, tramadol, magnesium sulfate, α2-agonists, physostigmine, doxapram, methyl phenidate, 5-HT3 antagonists, and the like.

In a preferred embodiment, the control unit is configured to provide output signals to a drug delivery device adapted to administer said anti-shivering medication, where the delivery device is not necessarily part of the overall device.

A drug delivery device includes any means for containing and releasing a drug, wherein the drug is released to a subject. The term “drug delivery device” refers to any means for containing and releasing a drug, wherein the drug is released into a subject. The means for containing is not limited to containment in a walled vessel, but may be any type of containment device, including non-injectable devices (pumps etc.) and injectable devices, including a gel, a viscous or semi-solid material or even a liquid. Drug delivery devices may be inhaled, oral, transdermal, parenteral and suppository. Inhaled devices include gaseous, misting, emulsifying and nebulizing bronchial (including nasal) inhalers; oral includes mostly pills; whereas transdermal includes mostly patches. Parenteral includes injectable and non-injectable devices. Non-injectable devices may be “implants” or “non-injectable implants” and include e.g., pumps and solid biodegradable polymers. Injectable devices are split into bolus injections, that are injected and dissipate, releasing a drug all at once, and depots, that remain discrete at the site of injection, releasing drug over time. Depots include e.g., oils, gels, liquid polymers and non-polymers, and microspheres. Many drug delivery devices are described in Encyclopedia of Controlled Drug Delivery (1999), Edith Mathiowitz (Ed.), John Wiley & Sons, Inc. The term “drug” as used herein, refers to any substance meant to alter animal physiology. The term “dosage form” refers to a drug plus a drug delivery device. The term “formulation” (or “drug formulation”) means any drug together with a pharmaceutically acceptable excipient or carrier such as a solvent such as water, phosphate buffered saline or other acceptable substance. A formulation may contain a drug and other active agents. It may also contain an excipient, solvent or buffer or stabilizing agent.

In another preferred embodiment, the control unit is configured to provide output signals to a drug delivery device which is part of the overall device. In other words, the device according to present invention comprises a drug delivery device and wherein the control unit is configured to provide output signals to said drug delivery device. Such device may be semi-automated or automated, such that when the shivering becomes excessive, the control unit automatically provides an output signal to the drug delivery device adapted to deliver anti-shivering medication to the patient without or with only minimal intervention of medical personnel.

In particular, the recommendation may comprise an instruction to raise the body surface temperature, also referred to herein as counter-warming. In contrast to core body temperature body which generally refers to the temperature of the internal environment of the body, including organs such as the heart and liver, body surface temperature generally refers to the temperature of the skin at various body parts, including limbs, hand, feet or extremities.

Such selective warming in shiver trigger areas can be accomplished by providing heat to warm surface regions of a person. This eliminates shivering while still allowing the desired or necessary cooling of body portions. Regions of the patient could be cooled and warmed at the same time in different areas of the body. The devices and methods disclosed herein may therefore be advantageously used to bring core temperature of the patient down. In one embodiment, warming may be applied under the arms, in the neck area, and other areas of the body, such as extremities, face, wrists, etc. A skilled person in the art is able to determine which body parts can be selectively warmed, such as disclosed in US Patent Application No. 20140135879.

In one embodiment, the device according to the present invention comprises a control unit which provides an instruction to raise the surface temperature at chest, extremity and/or limb temperature of the patient.

Preferably, the control unit is configured to provide output signals to a heating device which is not necessarily part of the device which controls the infusion fluid according to the present invention. The heating device can be engaged automatically or by medical personnel to reduce or minimize shivering.

For example, the heating device may be temperature adaption pad(s), preferably applied to the patient's skin, warming vests, head wraps, intravascular catheter(s), gas inhalation, transnasal evaporative catheter systems, extracorporal adaption of the blood temperature, temperature adaption mattress and/or blankets, temperature adaption tents, heart-lung machine, peritoneal-lavage systems, blood and fluid warmers etc.

The heating device may be based on a heat transfer, e.g. by convection and/or by conduction. The device may interact with a surface of the patient such as the skin of a patient, or an intravascular catheter which provides heat to the blood flowing around the catheter. In one preferred embodiment, the control unit is configured to provide output signals to a heating device adapted to raise the blood temperature of the patient.

However, the device preferably comprises a heating device, wherein the control unit is configured to provide output signals to a heating device intended to raise the body surface temperature of the patient. For example, the heating device may comprise heat pads or wraps adapted to raise the surface temperature. Pads and wraps can be placed under the patient, wrapped around the desired portion of the body, such as the neck, armpits, chest, abdomen, etc., for easier placement and use. Various types of fasteners may be used to hold the pad(s) in place, such as hook and loop, ties, snaps, etc. Preferably, the heat pads are adapted to raise the surface temperature at the chest, limb, and/or extremity of the patient.

In one embodiment, the control unit is configured to provide an instruction to raise the body surface temperature of the patient or output signals to a heating device while providing an output signal to the flow control unit to maintain the flow rate and/or the temperature of the infusion fluid.

In one preferred embodiment, the heating device is a thermal blanket which may or may not be inflatable. The blanket could be constructed to warm the patient convectively by exhausting warm air onto the patient, for example as described in U.S. Pat. No. 8,105,370.

Should body temperature exceed the desired therapeutic body temperature or in case of over-warming by the heating device, the flow rate may be increased, or the temperature of the infusion fluid may be decreased to maintain the desired therapeutic body temperature.

Thus, in one preferred embodiment, the control unit is configured to provide a signal to the flow control unit which increases cooling power of the infusion fluid, for example by increasing the flow rate and/or reducing the temperature of the infusion fluid.

In a preferred embodiment, the control unit is configured to receive input signal indicating the level of shivering entered by a user. Preferably, the control unit prompts a user to enter the input signal. In some embodiments, the control unit is configured to receive input signals that are entered by a doctor or other caretaker. In such embodiments, the device comprises a user interface with a user information output such as a screen, for prompting the user for input signals to be entered, suitably via a touchpad screen or keyboard. Various arrangements are possible and within the scope of the invention, for example, in some embodiments, the user is prompted at least whenever a fresh infusion bag is to be connected to the device and/or at regular time intervals. The input signals that are to be entered can, for example, be data defining which type of infusion fluid is connected, most suitably by choosing from a list stored in the memory of the device of typical conventional infusion fluids. Some common infusion fluids are defined above in a non-limiting list.

In yet another preferred embodiment, the input signal is provided by a sensor measuring the level of shivering. For example, electromyography (EMG) is sensitive to shivering. Electromyography (EMG) measures the small electrical current generated by the exchange of ions across muscle fibers during voluntary or involuntary muscle contractions. Muscle fibers are innervated by alpha motor neurons that cause contraction to occur when the action potential depolarization threshold of the motor nerve is reached. EMG electrodes measure the electromagnetic field generated by the depolarization as ionic-related voltage changes. Electromyography analysis has been shown to be useful in studying shivering, as it provides instantaneous information on the current state of the muscle. Shivering creates a unique characteristic EMG signal at 200-250 Hz with a 4-8 cycle/min waxing and waning pattern that correlates to early stages of shivering. Therefore, before visible or vigorous shivering occurs, EMG may be used as an early detection mechanism. Shivering is tonic and constant in frequency for periods up to 10 minutes. The shivering EMG signal can be obtained from multiple muscle groups with the intensity of signal varying with size of the muscle groups being measured. For example, EMG signals of the masseter and chest can be measured in order to quickly and accurately assess shivering. A device which detects and processes raw EMG signals obtained from surface EMG electrodes located on the masseter muscles and thorax (indicative of early stage shivering) can be used. An algorithm may be used in the controlling unit to reduce cooling or increase cooling to the individual/patient depending on readings from the EMG. If shivering is detected, either visibly or through the use of an electronic device such as EMG, one recommendation is to apply selective warming or a reduction in cooling. In one embodiment, selective application of heat to the chest and/or limbs may stop the shivering response upon detection. In other embodiments, temperature sensors, HR, EEG or index of brain function/alertness may be used as well to indicate the level of shivering. Electrocardiogram can also be used in the present invention. It is also known that during shivering, an artifact could be observed in electrocardiogram (EKG) which indicates the small muscle movements of early stage shivering (Graham et al., (2001). The electrocardiogram in hypothermia. Wilderness & Environmental Medicine, 12(4), 232-235. 2001).

Most preferably, the input signal is provided by a motion sensor that is used to measure the level of shivering. The sensor can be adapted to obtaining a signal from a muscle mass that is susceptible to shivering such as a direct motion detector, for example, an accelerometer that leads from the detector to a signal processor, or an indirect motion detector, for example, as one or more electrodes adapted for placement in or on a surface of the body of the patient that leads from the electrical connection of the electrodes to a signal processor. Motion sensor that is used to measure the level of shivering is known in the art. For example, US Patent Application No. 2009575708 discloses detecting the movement using a motion detector, an accelerometer, an electrical signal from one or more electrodes, an EMG signal or a combined ECG and EMG signal.

In one embodiment, the device may comprise a motion sensor and the control unit may be configured to receive input signal indicating the level of shivering from the motion sensor. In another embodiment, the device may comprise an ECG sensor and the control unit may be configured to receive input signal indicating the level of shivering from the ECG sensor. In yet another preferred embodiment, the device may comprise an EMG sensor and the control unit may be configured to receive input signal indicating the level of shivering from the EMG sensor. In another embodiment, the device may comprise a plurality of aforementioned sensors.

In another embodiment, the control unit can be configured to provide a signal to the flow control unit to adjust the temperature of the infusion fluid. In general, the control unit is preferably configured to receive input data indicating temperature of infusion fluid being administered and/or connected to the device. Accordingly, cooled infusion fluid can be administered with this invention and the device described herein. The cooled infusion fluid may be delivered between −1 to 14° C., such as −1° C., 0° C., 1° C., 2° C., 3° C., 4° C., 5° C., 6° C., 7° C., 8° C., 9° C., 10° C., 11° C., 12° C., 13° C., or 14° C. Infusion fluid is preferably delivered with a minimum temperature of 3.5° C., preferably 3.6° C., more preferably 3.7° C., more preferably 3.8° C., more preferably 3.9° C. and most preferably 4° C. and/or cooled infusion fluid is provided at a maximum temperature of 6° C., preferably 5.5° C., more preferably 5.0° C., more preferably 4.5° C., more preferably 4.25° C. and most preferably 4.0° C. The temperature of the infusion fluid can be decreased by 0.1° C., 0.2° C., 0.3° C., 0.4° C., 0.5° C., 0.6° C., 0.7° C., 0.8° C., 0.9° C., 1.0° C., 1.1° C., 1.2° C., 1.3° C., 1.4° C., 1.5° C., 1.6° C., 1.7° C., 1.8° C., 1.9° C., 2.0° C. or more. The control unit may further store data indicating the medication administered to the patient. The output signals generated by the control unit which indicates recommendation for therapy may be determined based on said data.

If the level of shivering continues for a prolonged period of time over a limit which is undesired for the patient, the control unit may be configured to automatically stop the infusion or to provide an output signal indicating a recommendation to stop infusion. For example, control unit may further store data indicating the anti/shivering medication or other medications already administered to the patient. If the patient has already received anti-shivering medication exceeding recommended limits, the control unit would refrain from providing recommendation which comprise an instruction to administer further anti-shivering medication. Instead, the control unit may send a signal to adjust the flow control unit or a recommendation which comprises an instruction to raise temperature of the patient, especially the surface body temperature.

In some embodiments, the control unit is configured to receive input signals from at least one sensor that indicates the level of shivering. In one preferred embodiment, the input signals may further comprise signals selected from the group consisting of signals indicating medical condition of patient, signals indicating desired therapeutic body temperature of patient, and signals indicating blood status of patient, signals indicating concentration of at least one electrolyte, signals indicating additional infusion fluid that the patient is being administered. Input signals that can be entered in the device and for which the device may prompt the user may be selected from but are not limited to one or more of the following: signal indicating concentration of at least one electrolyte, signal indicating additional infusion fluid that the patient is being or is to be administered, signal indicating medication that the patient is being administered or has received, signal indicating medical condition of patient, signal indicating desired therapeutic body temperature of patient, and signal indicating blood status of patient. “Blood status” in this context may refer to any of various parameters describing status of blood, such as hemoglobin value, platelet count, etc. “Medical condition” in the context herein may refer to any vital signal such as but not limited to pulse, blood pressure, body temperature, or other relevant input parameter defining medical condition. In useful embodiments, the device is connected to one or more temperature sensors that provide the control unit with values indicating the body temperature of the patient.

In other embodiments, the control unit is configured to receive input signals directly (without user input) from sensors, such as but not limited to sensors for sensing vital signals or other patient signals (e.g. heart rate, blood pressure, breathing rhythm . . . etc.). In some embodiments, the control unit is able to receive a combination of input signals, both entered manually and received from sensors and/or external computers, systems, etc. Such signals can be used by the control unit for providing recommendations. For example, such sensors may detect an adverse event, such as heart rate irregularities, which may indicate lack of potassium, then the control unit can respond to by giving an output signal with instructions to change the infusion fluid to a fluid with higher potassium content. This can happen either such that the device will start administering potassium containing fluid instead of or in addition to non-potassium fluid, or by providing output signals to a user instructing to change infusion fluid.

The device may further comprise a user interface which prompts the user for input information and outputs visual information indicating said output signals.

The device of the present invention may suitably be arranged also with means to monitor and/or adjust volume being administered, by controlling flow rate of IV fluid and monitoring fluid loss from the patient, by suitable sensors or prompting for relevant data to be entered representing fluid loss. Accordingly, in some embodiments of the invention, the device is configured to adjust the volume of one or more medical infusion fluids, the device comprising at least one first determining unit adapted to measure and/or determine the volume of the medical infusion fluid flowing through a delivery duct and adapted to provide a respective first signal; at least one second determining unit adapted to measure and/or determine the volume and/or weight of at least one released body fluid and/or a physiological parameter and further adapted to provide a respective second signal; and at least one volume controlling unit adapted to control the flow of the medical infusion fluid through the delivery duct on the basis of the first and the second signals.

Intracranial pressure (ICP) is the pressure inside the skull and thus in the brain tissue and cerebrospinal fluid (CSF). Increased intracranial pressure (ICP) is one of the major causes of secondary brain ischemia that accompanies a variety of pathological conditions, most notably, traumatic brain injury (TBI), stroke, and intracranial hemorrhages. In some embodiments, the control unit of the device is further configured to receive input signals indicating intracranial pressure (ICP) and optionally blood pressure of a patient and to provide output signals indicating one or more recommendations for therapy based on said received input signals. Thus, the device can, in such embodiments, aid in the treatment of patients with elevated ICP. The input signals may be provided by a user, from an external computer system, or internally from a component of the device. The control unit may be configured to receive input signals indicating the level of intracranial pressure. ICP can be measured with invasive or noninvasive methods. Invasive methods normally require an insertion of an ICP sensor into the brain ventricle or parenchymal tissue. ICP can also be measured non-invasively. Several methods for noninvasive measuring of elevated ICP have been proposed: radiological methods including computed tomography and magnetic resonance imaging, transcranial Doppler, electroencephalography power spectrum analysis, and the audiological and ophthalmological techniques. In one embodiment, the recommendation provided by the control unit comprises an instruction to administer ICP-reducing medication. As used herein, the term ICP-reducing medication is intended to mean any biologically active agent or drug or combination of agents or drugs that is administered to a patient for the purpose of reducing ICP. Any ICP-reducing agents can be used, such as agents commonly used in hyperosmolar therapy such as mannitol. In a preferred embodiment, the control unit is configured to provide output signals to a drug delivery device adapted to administer said ICP-reducing medication, where the delivery device is not part of the overall device.

In another preferred embodiment, the control unit is configured to provide output signals to a drug delivery device which is part of the overall device. In other words, the device according to present invention comprises a drug delivery device and wherein the control unit is configured to provide output signals to said drug delivery device. Such device may be semi-automated or automated, such that when the ICP is over a given limit or range, the control unit automatically provides an output signal to the drug delivery device adapted to deliver ICP-reducing medication to the patient without or with only minimal intervention of medical personnel.

Preferably, the device disclosed in the present invention is adapted to be mounted into a rack. There is generally a need to standardize the different components used in hospitals and being placed next to patients for the delivery of different medications. Moreover, racks with a plurality of components for medical devices become more and more used. Typically, they offer a slot for introducing medical devices such as syringes or pumps pumping infusion fluids with different medicaments for their intravenous delivery. For example, in U.S. Pat. No. 4,756,706 a central processing unit is described for centrally controlling and monitoring the different components mentioned before. WO 2013/102495 concerns an arrangement of a rack and a medical device to be attached to the rack. Thus, an arrangement of a rack and a medical device may be advantageously provided to allow for an easy attachment of the medical device to the rack, by providing a secure and reliable and at the same time versatile electrical connection between the medical device and the rack. Thus, in one preferred embodiment, the device is adapted to be mounted into a rack. For example, a frame can be provided and assembled and/or adapted to introduce the device into one or more slots of a rack of given dimension and/or shape. The rack can be of any shape, either with a single post for assembling the medical device and other medical devices and/or a shelf-like structure with more than one post and/or at least one or more walls. Moreover, the rack can be adapted to allow several medical devices to be placed in at least one vertical and/or at least one horizontal row(s). The frame of the medical device comprises at least one, preferably at least two rail(s) and/or hook(s) for inserting the device into the rack. In case the rack comprises essentially one post, one or more hooks can be provided. In case of a shelf-like rack one or more rail(s) can be provided for slidingly placing or allowing the device to be placed in the rack. The rail(s) and/or the respective counterpart(s) at the rack can be of any known structure with sliding or bearing-supported structure(s) and/or of expandable and/or telescoping nature. Moreover, the medical device can comprise at least one releasable lock for releasably locking the device in the rack. This can prevent the accidentally removing of the medical device in or at the rack and/or the defined position of the device and/or its other components interfering with the rack and other elements. The term locking does not necessarily mean that a specific element is provided it can also be enabled by a mechanical and/or electronic indicator indicating the defined position of the medical device at and/or in the rack. The present invention also provides a rack which includes one or more of the devices described herein.

Target temperature for hypothermia ranged is between 33.0° C. and 35.5° C., depending on the specific condition being treated, preferably 33.0° C., 33.5° C., 34.0° C., 34.5° C., 35.0° C. or 35.5° C. Cooling is achieved with a device as described in the present application. Decisions regarding the method and duration of cooling may be under the discretion of the attending neurointensivist or another medical professional.

Because untreated shivering may negate the beneficial effects of UM, the BSAS is monitored at least hourly, while more frequently during the induction of cooling and the rewarming phase after mild hypothermia when shivering is more likely to occur. The patient's neck, masseter, and chest muscles is palpated to determine if the patient is shivering. The absence of any shivering in this part of the body is scored as 0. Shivering visible on the neck and thorax is scored as 1. Moderate shivering includes shivering of the neck and chest muscles and involvement of the upper extremities and is scored as 2. Movement of the neck, chest, and all extremities indicates severe shivering, a BSAS score of 3. The attending neurointensivist or another medical professional can enter the level of shivering into the device of the present application.

When the device receives input signals indicating the level of shivering of the patient which corresponds to score 2 or 3 in BSAS scale, the device provides output signals indicating a recommendation which is an instruction to administer anti-shivering medication.

The medication can comprise meperidine (Demerol) which is able to decrease both shivering and the vasoconstriction. In one embodiment, a combination of low-dose buspirone (Buspar, 20 mg) and low-dose meperidine (25 mg) may be used to decrease the shivering. In another embodiment, dexmedetomidine (Precedex) plus meperidine can be used to reduce the shivering, with doses ranging from 0.2 to 1.5 μg/kg per hour. Patients that have seizures or renal failure, or that take monamine oxidase inhibitors are preferably not given meperidine because it may decrease the seizure threshold in some patients. In patients with bradycardia, dexmedetomidine may not be the drug of choice because it can slow the heart rate further, leading to an unstable hemodynamic status.

If shivering cannot be abolished with the previously mentioned agents, the device provides output signals indicating a recommendation which is an instruction to administer neuromuscular blockade with medications such as vecuronium (Norcuron). Patients given a neuromuscular blocker should be receiving mechanical ventilation and concurrent sedatives or analgesics.

In another embodiment, the recommendation is provided according to standard procedures such as the Columbia Anti-Shivering Protocol (Choi et al., Neurocrit Care 2011 Jun. 14(3):389-394) or as shown in Table 3.

TABLE 3

| Step | | Intervention | Dose |
|---|---|---|---|
| 0 | Baseline | Acetaminophen | 650-1000 mg every 4-6 h |
| | | Buspirone | 30 mg every 8 h |
| | | Magnesium sulfate | 0.5-1 mg/h i.v. Goal |
| | | Counterwarming | (3-4 mg/dl) |
| | | | 43° C./MAX Temperature |
| 1 | Mild sedation | Dexmedetomidine | 0.2-0.5 μg/kg/h |
| | | or Opioid | Fentanyl starting dose |
| | | | 25 μg/h |
| | | | Meperidine or Pethidine |
| | | | 50-100 mg i.m. or i.v. |
| 2 | Moderate | Dexmedetomidine | Doses as above |
| | sedation | and Opioid | |
| 3 | Deep sedation | Propofol | 50-75 μg/kg/min |
| 4 | Neuromuscular | Vecuronium | 0.1 mg/kg i.v. |
| | blockade | | |

At baseline (Step 0), series of interventions prior to the initiation of cooling can be taken to minimize shivering during the induction phase. These measures can be continued empirically throughout the entire cooling period. The initial intervention (Step 1) of either an opiate or dexmedetomidine may be made for any patient demonstrating moderate to severe shivering (BSAS score 2-3) despite all baseline interventions. The choice between which agent to use first may be based upon additional needs for the particular patient. For example, opiates can be considered first in patients with either poorly controlled pain or baseline bradycardia, and likewise, dexmedetomidine in patients with poorly controlled agitation. If the initial intervention is not successful, the next step may be the combination of dexmedetomidine and an opiate (Step 2). Both steps 1 and 2 of the protocol preferably maximize the use of one agent prior to proceeding to the second agent; therefore, patients graduating between Steps 1 and 2 have a synergistic anti-shivering effect of an opiate and dexmedetomidine. Deep sedation with propofol may be achieved in those patients failing to achieve adequate shiver control (BSAS score 1) (Step 3). As oftentimes patients are already on a lower dose of propofol for sedation, only increases in the initial dose of propofol or high levels of propofol were considered as shivering interventions. Neuromuscular blockade with boluses of a paralytic is reserved for hypothermic patients not able to achieve control with deep sedation (Step 4). In one particularly preferred embodiment, the patient is given paracetamol 1000 mg i.v. and is covered in thermal blanket prior to the initiation of cooling. When level of shivering exceeds BSAS score 2, the device provides an output signal to recommend the following medication: pethidine 1 mg/kg/KG i.v. (max. 75 mg), dexmedethomidine, buspiron 30 mg p.o.

The device of the invention is preferably configured so as to fit in a conventional hospital rack system, i.e. a bedside rack for containing one or more modular devices for patient care and/or monitoring. In such embodiments the device is configured and designed as a modular unit to fit in such rack. The device can in certain such embodiments comprise more than modular unit, for example when it is desired to actively cool the infusion fluid by keeping in a cooled storage compartment while the fluid is administered such cooling compartment can be as an add-on module.

### PREFERRED EMBODIMENTS

The present invention will become more fully understood from the description before and particularly below and the accompanying drawings that are given by way of illustration only and show and/or exemplify preferred aspects thereof and wherein

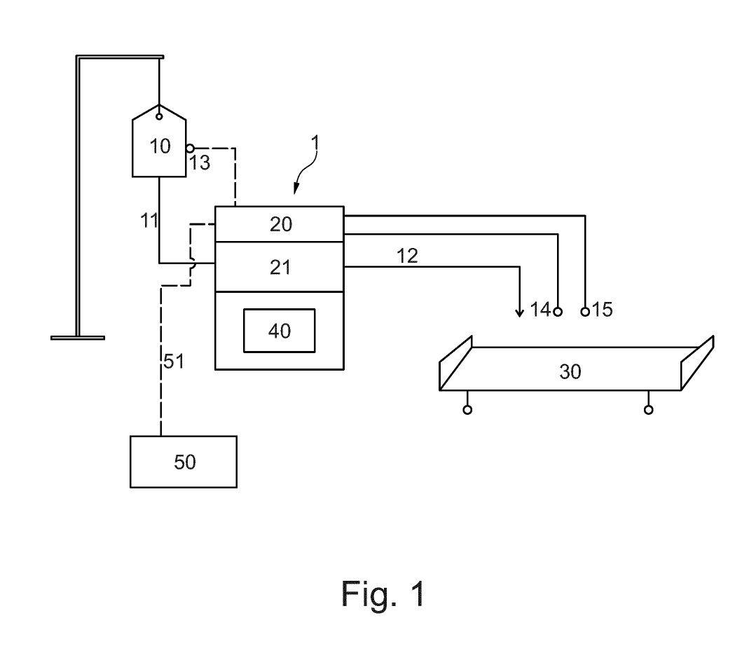

FIG. 1 illustrates an embodiment of the configuration of the device of the invention.

The device 1 as shown in FIG. 1 comprises a control unit 20, a flow control unit 21 and an input/output screen 40. A typical infusion fluid bag 10 is hung on a conventional supporting device. From the bag, a duct 11 provides infusion fluid through the flow control unit 21, which passes the infusion fluid onwards to a patient (not shown) through duct 12. An optional input line 51 from an external computer 50 is shown. Adjacent to the bag 10 is a barcode scanner 13 for detecting and registering type of IV fluid bag, providing a signal to the control unit. Alternatively and optionally, input data concerning the type of IV fluid bag is input via the input screen 40.

Motion sensors 14 and temperature sensor 15 are shown. They can be applied to the patient to measure the level of shivering and the core body temperature.

Bed 30 is also shown to schematically indicate the patient.

As used herein, including in the claims, singular forms of terms are to be construed as also including the plural form and vice versa, unless the context indicates otherwise. Thus, it should be noted that as used herein, the singular forms “a,” “an,” and “the” include plural references unless the context clearly dictates otherwise.

Throughout the description and claims, the terms “comprise”, “including”, “having”, and “contain” and their variations should be understood as meaning “including but not limited to”, and are not intended to exclude other components.

The present invention also covers the exact terms, features, values and ranges etc. in case these terms, features, values and ranges etc. are used in conjunction with terms such as about, around, generally, substantially, essentially, at least etc. (i.e., “about 3” shall also cover exactly 3 or “substantially constant” shall also cover exactly constant).

The term “at least one” should be understood as meaning “one or more”, and therefore includes both embodiments that include one or multiple components. Furthermore, dependent claims that refer to independent claims that describe features with “at least one” have the same meaning, both when the feature is referred to as “the” and “the at least one”.

It will be appreciated that variations to the foregoing embodiments of the invention can be made while still falling within the scope of the invention. Alternative features serving the same, equivalent or similar purpose can replace features disclosed in the specification, unless stated otherwise. Thus, unless stated otherwise, each feature disclosed represents one example of a generic series of equivalent or similar features.

Use of exemplary language, such as “for instance”, “such as”, “for example” and the like, is merely intended to better illustrate the invention and does not indicate a limitation on the scope of the invention unless so claimed. Any steps described in the specification may be performed in any order or simultaneously, unless the context clearly indicates otherwise.

All of the features and/or steps disclosed in the specification can be combined in any combination, except for combinations where at least some of the features and/or steps are mutually exclusive. In particular, preferred features of the invention are applicable to all aspects of the invention and may be used in any combination.

1. The device for controlling and managing administration of infusion fluid for temperature regulation therapy, comprising:
at least one flow control unit for regulating flow rate and/or temperature of infusion fluid, and
at least one control unit for receiving input signals and providing output signals,
wherein the control unit is configured to receive input signals indicating the level of shivering of the patient and to provide output signals indicating one or more recommendations for therapy based on said received input signals.

2. The device according to claim 1, wherein the control unit is configured to receive input signals from at least one external computer system.

3. The device according to claim 1, wherein the control unit is configured to receive input data indicating body temperature of patient, such as core body temperature or body surface temperature.

4. The device according to claim 1, wherein the control unit is configured to provide a signal to the flow control unit.

5. The device according to claim 1, wherein the control unit is configured to provide a signal to the flow control unit to reduce flow rate or increase temperature of the infusion fluid.

6. The device according to claim 1, wherein the recommendation comprises an instruction to administer anti-shivering medication.

7. The device according to claim 6, wherein the anti-shivering medication comprises opiates, tramadol, magnesium sulfate, α2-agonists, physostigmine, doxapram, methylphenidate, and/or 5-HT3 antagonists.

8. The device according to claim 1, wherein the control unit is configured to provide output signals to a drug delivery device adapted to administer said anti-shivering medication.

9. The device according to claim 1, further comprising a drug delivery device, and wherein the control unit is configured to provide output signals to said drug delivery device.

10. The device according to claim 1, wherein the recommendation comprises an instruction to raise surface temperature of the patient.

11. The device according to claim 1, wherein the control unit is configured to provide output signals to a heating device.

12. The device according to claim 1, further comprising a heating device, and wherein the control unit is configured to provide output signals to a heating device.

13. The device according to claim 10, wherein the heating device comprises heat pads adapted to raise the surface temperature of the patient.

14. The device according to claim 1, wherein the control unit is configured to provide an instruction to raise surface temperature of the patient or provide an output signals to a heating device while providing an output signal to the flow control unit to maintain the flow rate and/or the temperature of the infusion fluid.

15. The device according to claim 1, wherein the control unit is configured to provide a signal to the flow control unit to increase the flow rate or reduce the temperature of the infusion fluid.

16. The device according to claim 14, wherein the control unit is configured to increase the flow rate or reduce the temperature of the infusion fluid based on the signal indicating the body temperature and the desired therapeutic body temperature of the patient.

17. The device according to claim 1, wherein the control unit is configured to receive input signal indicating the level of shivering entered by a user, and, optionally, wherein the control unit prompts a user for input signals to be entered.

18. The device according to claim 1, wherein the control unit is configured to receive input signal indicating the level of shivering from a motion sensor, and wherein the device optionally comprises the motion sensor.

19. The device according to claim 1, wherein the control unit is configured to receive an input signal indicating the level of shivering from an ECG sensor and/or EMG sensor, and wherein the device optionally comprises the ECG sensor and/or the EMG sensor.

20. The device according to claim 1, wherein the control unit is configured to receive input data indicating temperature of infusion fluid being administered and/or connected to the device.

21. The device according to claim 1, wherein the control unit further stores data indicating the medication administered to the patient, and wherein said recommendation is determined based on said data.

22. The device according to claim 1, wherein the input signals comprise signals selected from the group consisting of signals indicating medical condition of patient, signals indicating desired therapeutic body temperature of patient, and signals indicating blood status of patient, signals indicating concentration of at least one electrolyte, signals indicating additional infusion fluid that the patient is being administered.

23. The device according to claim 1, comprising a user interface which prompts the user for input information and outputs visual information indicating said output signals.

24. The device according to claim 1, wherein the control unit is configured to receive input signals indicating intracranial pressure (ICP) and optionally blood pressure of a patient and to provide output signals indicating one or more recommendations for therapy based on said received input signals.

25. The device according to claim 1, wherein the control unit is configured to receive input signals that define infusion fluid to be administered or which is being administered, and store such information, and provide output signals to control electrolyte content of the infusion fluid based on said received input signals.

26. The device according to claim 1, further comprising
a. at least one first determining unit adapted to measure and/or determine the volume of the medical infusion fluid flowing through a delivery duct and adapted to provide a respective first signal,
b. at least one second determining unit adapted to measure and/or determine the volume and/or weight of at least one released body fluid and/or a physiological parameter and further adapted to provide a respective second signal, and
c. at least one volume controlling unit adapted to control the flow of the medical infusion fluid through the delivery duct on the basis of the first and the second signals, and optionally,
wherein the second determining unit is adapted to measure and/or determine the volume and/or weight of at least one released body fluid selected from urine, sweat, wound liquid, blood, breath vapors, evaporation and/or liquid content of stools.

27. The device according to claim 1 adapted to be mounted into a rack.

28. A rack comprising the device according to claim 1.

29. A method of controlling and managing administration of infusion fluid for temperature regulation therapy, comprising:
regulating flow rate and/or temperature of infusion fluid, and
receiving input signals and providing output signals,
wherein the input signals indicate the level of shivering of the patient,
providing output signals indicating one or more recommendations for therapy based on said received input signals.

30. A method of treating a mammal comprising using the method according to claim 29.
